(19) United States
(12) Patent Application Publication
(10) Pub. No.: US 2007/0275403 A1
(43) Pub. Date: Nov. 29, 2007

(54) Diagnostic methods for determining treatment

(76) Inventors: Larry E. Morrison, Glen Ellyn, IL (US); John S. Coon, Oak Park, IL (US)

Correspondence Address: Kevin M. Farrell;Pierce Atwood, Suite 350, One New Hampshire Avenue, Portsmouth, NH, 03801, US

(21) Appl. No.: 11/800,875

(22) Filed: May 8, 2007

Related U.S. Application Data
(60) Provisional application No. 60/799,216, filed on May 10, 2006.

(51) Int. Cl. C12Q 1/68

(52) U.S. Cl. 435/6

(57) ABSTRACT

The present invention provides methods for identifying cancer patients susceptible to effective treatment with inhibitors of the tyrosine kinase activity of EGFR. The invention is based on the discovery that polysomy of chromosome 7 can be used to selectively identify cancer patients that are likely to be successfully treated with EGFR tyrosine kinase inhibitors or agents that otherwise function similarly to tyrosine kinase inhibitors. The invention is based on the use of nucleic acid technology where nucleic acid probes are allowed to hybridize to cell samples and the number of copies of particular genetic regions quantified. The methods for identifying cancer patients of the invention can be enhanced by determination of expression of pAKT protein in patient samples. The invention also contemplates the treatment of those patients with tyrosine kinase inhibitors.

### BACKGROUND OF THE INVENTION

A host of cancers result in patient death every year and there continues to be a search for effective therapeutic drugs for treating cancer patients. In general, cancer survival is considered to be the most important measure of a therapeutic drug's effectiveness. For a cancer such as lung cancer, for which overall survival is relatively short (overall median survival less than 1 year in advanced cases), final approval of a drug in the United States by the FDA requires the demonstration of a significant association with patient survival. Significant association with response can bring approval in the short term, but patient follow up and eventual demonstration of significant association with survival is ultimately required.

Lung, colon and head and neck cancers account for a substantial proportion of cancer deaths. Lung cancer alone accounted for almost one third of cancer deaths in 2005. Non small cell lung cancer (NSCLC) comprises 80-85% of lung cancer cases in the United States. To improve on conventional chemotherapy, novel molecular agents designed to exploit non-lethal genetic and epigenetic alterations in cancer cells have been investigated as treatment strategies. One class of such therapeutic agents, the tyrosine kinase inhibitors (TKI), specifically targets receptor and non-receptor tyrosine kinases that control cell survival and proliferation. The success of TKI treatments such as the small molecule imatinib in chronic myelogenous leukemia and gastrointestinal stromal tumors supported application of TKIs to lung cancer, where the tyrosine kinase of the epidermal growth factor receptor (EGFR) is abnormally expressed.

Based on its central role in tumor progression, results from in vitro studies, and its aberrant expression in 40-80% of NSCLC, EGFR is an attractive target for therapeutic intervention. Agents targeting the tyrosine kinase activity of the EGFR protein, including gefitinib (Iressa, AstraZeneca) and erlotinib (Tarceva, OSI Pharmaceuticals), were expected to have significant efficacy in NSCLC. Clinically, however, gefitinib demonstrated limited success with response rates of 18.4% and 11.8% reported in phase II trials. Erlotinib produced a response rate of 12.3% in patients previously screened for EGFR expression. In a subsequent phase III trial gefitinib demonstrated an 8-13% response rate but no significant survival benefit.

Analysis of patient sub-populations revealed that female patients, Asian patients, non-smokers and those with bronchoalveolar/adenocarcinoma were more likely to respond to the TKI.

Additionally, a number of molecular characteristics have been assayed for association with response and predictive value for survival. These include increased expression of EGFR and related receptors, status of downstream factors and EGFR associated polymorphisms. Increased copy number of EGFR and HER2 genes (amplification or polysomy) detected by fluorescence in situ hybridization (FISH) and pAKT expression, have shown the best predictive value in several studies. The level of amplification and polysomy of EGFR can be determined using various nucleic acid probes directed to the EGFR gene and human chromosome 7. See, e.g., WO/2005/117553 A2 by the Regents of the University of Colorado. However, these teachings do not provide useful information regarding survival benefit.

Activating mutations in the kinase domain of EGFR are most highly correlated with response to TKI. The mutations were discovered through extensive sequence analysis of the EGFR gene which revealed that deletion of a conserved amino acid sequence, (E)LREA, in exon 19 and point mutations G719C in exon 18 and L858R in exon 21 of the EGFR gene correlated with response to gefitinib. Although later studies reported mutations in exon 20, the majority of the known EGFR mutations are in exons 19 and 21 with 50% in exon 19, 40% in exon 21, 5-10% (or less) in exon 18 and 6% in exon 20. Although mutations in exons 19 and 21 are associated with response to TKI, the exon 19 deletion mutations may be more highly correlated with lengthened survival than the exon 21 L858R mutation.

Like other biomarkers, the relationship between EGFR tyrosine kinase domain mutations and TKI efficacy is not absolute, in that response occurs in the absence of mutation and some tumors with mutations progress in spite of TKI therapy. Furthermore, particular mutations may not predict increased survival benefit with TKI therapy.

Thus, there continues to be a need for improved patient selection criteria based on molecular indices for application of targeted TKI and other such therapies.

### SUMMARY OF THE INVENTION

The present invention provides methods for identifying cancer patients susceptible to effective treatment (e.g., longer survival) with inhibitors of the tyrosine kinase activity of EGFR such as the small molecules gefitinib and erlotinib and the anti-EGFR monoclonal antibody cetuximab (Erbitux), and agents that function similarly to such inhibitors. The invention is particularly beneficial for identifying lung cancer patients, particularly NSCLC patients expected to obtain survival benefit from TKIs. The invention is based on the discovery that detection of abnormal copy number of human chromosome 7 (aneusomy or, preferably, polysomy of chromosome 7) in patients can be used to selectively identify cancer patients that are likely (or unlikely) to be successfully treated with TKIs for EGFR such as gefitinib, erlotinib and cetuximab and agents that function similarly to TKIs. Relative to other markers frequently associated with cancer, Applicants have found that abnormal copy number of chromosome 7 is the most useful single marker predicting increased survival time.

This aspect of the invention is based on the use of nucleic acid probe technology where nucleic acid probes are allowed to hybridize to patient samples and the number of copies of particular genetic regions quantified. Preferably, in situ hybridization and, more preferably, fluorescent in situ hybridization (FISH) with fluorescently labeled nucleic acid probes is used. The hybridization results are then used to determine the likelihood that the patient will be treated successfully with a TKI. Preferably, the patients are NSCLC patients and the samples are lung cell samples.

The methods of the invention can be used with other markers used to evaluate patients relative to treatment with TKIs. For example, the detection of abnormal copy number of chromosome 7 can be combined with detection of gain and/or polysomy of epidermal receptor growth factor receptor gene and/or detection of gain and/or polysomy of the HER2 gene to better inform the identification of cancer patients that are likely (or unlikely) to be successfully treated with TKIs.

Further aspects of the invention include detection of the level of expression of associated biological markers such as phosphorylated-Akt or PTEN proteins. The expression level of pAKT and PTEN can be determined by well known immunohistochemical techniques. Patients whose samples exhibit abnormal copy number of chromosome 7 and expression of such proteins are likely to be good candidates for treatment with TKIs.

The methods for identifying candidate patients for treatment with TKIs to EGFR comprise: a) obtaining a biological sample comprising cells from a patient suspected of having a carcinoma; b) contacting the sample with a chromosomal probe able to detect the presence of chromosome 7, under hybridization conditions; c) determining whether the sample has abnormal copy number of chromosome 7 and d) identifying the candidate as being suitable for treatment. Preferably, the method comprises the step of determining whether the sample has polysomy of chromosome 7. Typically, probes able to detect the presence of chromosome 7 allow enumeration of the chromosome. Examples of such are probes designed to specifically hybridize to the centromere of chromosome 7 (CEN 7 probes). The candidate patient may only be suspected of having cancer cells. The candidate patient may also have been previously diagnosed as having cancer cells from diseases including, but not limited to, lung, colon, and head and neck cancers and other cancers. Preferably, the cancer is NSCLC.

The methods of the invention may further comprise contacting a biological sample (e.g., a tissue sample) comprising the cells from the candidate patient with expression reagents such as antibody probes that specifically bind proteins such as phosphorylated AKT (pAKT) or PTEN.

The present invention also contemplates kits and sets of probes for use in diagnosing and treating cancers, and preferably methods for determining the susceptibility of patients suspected of having cancer to successful treatment with inhibitors of the tyrosine kinase activity of the EGFR protein. Preferably, fluorescently labeled probes are used and included in the probe sets and kits. The kits and probe sets comprise probes able to detect the copy number for chromosome 7. Kits may also include reagents for carrying out the methods of the invention, such as reagents for measuring expression. Reagents for IHC include antibody probes that specifically bind to proteins such as pAKT or PTEN, reagents to block non-specific binding of the antibody to the slide, various buffers for washing the slide, and detection reagents.

### DETAILED DESCRIPTION OF THE INVENTION

The invention includes methods for identifying candidate patients for treatment with inhibitors of the tyrosine kinase activity of EGFR such as the small molecules gefitinib and imatinib or the antibody cetuximab and the treatment of such patients with such inhibitors. The invention also includes methods for identifying candidate patients for treatment with agents that function similarly to inhibitors of the tyrosine kinase activity of EGFR and the treatment of such patients with such agents. Preferably, the patients are NSCLC patients and the inhibitor is gefitinib or imatinib.

The identification of a candidate patient (e.g., a NSCLC cancer patient) for treatment with inhibitors of the tyrosine kinase activity of EGFR (TKIs) can be determined by identifying chromosomal aberrations in an appropriate biological sample obtained from the patient. This can be accomplished by in situ hybridization to establish the presence of aneusomy of chromosome 7 in the patient sample. In general, in situ hybridization typically includes the steps of fixing a biological sample, hybridizing a chromosomal probe to target DNA contained within the fixed sample, washing to remove non-specifically bound probe, and detecting the hybridized probe. The in situ hybridization can also be carried out with the specimen cells in liquid suspension, followed by detection by flow cytometry.

Identification of patients for treatment with TKIs and similar agents may be enhanced by evaluating the expression of suitable proteins such as pAKt and PTEN. Patients whose samples are found with expression of such proteins in conjunction with abnormal copy number of chromosome 7 are likely to be good candidates for treatment with TKIs.

Chromosomal Probes. Suitable probes for use in the in situ hybridization methods utilized with the invention for the detection of abnormal copy number (aneusomy or, preferably, polysomy) of chromosome 7 are typically chromosome enumeration probes. These are probes that hybridize to a chromosomal region, usually a repeat sequence region, and indicate the presence or absence of chromosome 7. As is well known in the art, a chromosome enumeration probe can hybridize to a repetitive sequence, located either near or removed from a centromere, or can hybridize to a unique sequence located at any position on a chromosome. For example, a chromosome enumeration probe can hybridize with repetitive DNA associated with the centromere of a chromosome. Centromeres of primate chromosomes contain a complex family of long tandem repeats of DNA comprised of a monomer repeat length of about 171 base pairs that are referred to as alpha-satellite DNA. A non-limiting example of a specific chromosome enumeration probe is the SpectrumGreen™ CEP® 7 probe (Abbott Molecular Inc.) for chromosome 7 described in the Examples.

Probes for detecting copy number of chromosome 7 can be used in conjunction with probes for detecting other specific markers to better inform the decision whether to treat the patient with TKIs. For example, the detection of polysomy of chromosome 7 can be combined with locus specific probes to determine the status of amplification and/or polysomy of the EGFR gene and/or the HER-2 gene. A locus specific probe hybridizes to a specific, non-repetitive locus on a chromosome. Probes useful to determine the status of amplification and/or polysomy of the EGFR gene and the HER-2 gene include the Vysis LSI EGFR SpectrumOrange and the LSI HER-2 SpectrumGreen probes, respectively (Abbott Molecular Inc.). Chromosome arm probes, i.e., probes that hybridize to a chromosomal region and indicate the presence or absence of an arm of a specific chromosome, may also be useful.

Probes that hybridize with centromeric DNA are available commercially from Abbott Molecular Inc. (Des Plaines, Ill.) and Molecular Probes, Inc. (Eugene, Oreg.). Alternatively, probes can be made non-commercially using well known techniques. Sources of DNA for use in constructing DNA probes include genomic DNA, cloned DNA sequences such as bacterial artificial chromosomes (BAC), somatic cell hybrids that contain one or a part of a human chromosome along with the normal chromosome complement of the host, and chromosomes purified by flow cytometry or microdissection. The region of interest can be isolated through cloning or by site-specific amplification via the polymerase chain reaction (PCR). See, for example, Nath, et al., Biotechnic Histochem, 1998, 73 (1): 6-22; Wheeless, et al., Cytometry, 1994, 17:319-327; and U.S. Pat. No. 5,491,224. Synthesized oligomeric DNA or peptide nucleic acid (PNA) probes can also be used.

The size of the chromosomal region detected by the probes used in the invention can vary, for example, from the alpha satellite 171 base pair probe sequence noted above to a large segment of 900,000 bases. Locus-specific probes that are directly labeled are preferably at least 100,000 bases in complexity, and use unlabeled blocking nucleic acid, as disclosed in U.S. Pat. No. 5,756,696, herein incorporated by reference, to avoid non-specific binding of the probe. It is also possible to use unlabeled, synthesized oligomeric nucleic acid or protein nucleic acid as the blocking nucleic acid.

Chromosomal probes can contain any detection moiety that facilitates the detection of the probe when hybridized to a chromosome. Effective detection moieties include both direct and indirect labels as described herein. Examples of detectable labels include fluorophores (i.e., organic molecules that fluoresce after absorbing light), radioactive isotopes (e.g., ³²p, and ³H) and chromophores (e.g., enzymatic markers that produce a visually detectable marker). Fluorophores are preferred and can be directly labeled following covalent attachment to a nucleotide by incorporating the labeled nucleotide into the probe with standard techniques such as nick translation, random priming, and PCR labeling. Alternatively, deoxycytidine nucleotides within the probe can be transaminated with a linker. The fluorophore can then be covalently attached to the transaminated deoxycytidine nucleotides. See, e.g., U.S. Pat. No. 5,491,224 to Bittner, et al., which is incorporated herein by reference. Useful probe labeling techniques are described in Molecular Cytogenetics: Protocols and Applications, Y.-S. Fan, Ed., Chap. 2, “Labeling Fluorescence In Situ Hybridization Probes for Genomic Targets”, L. Morrison et. al., p. 21-40, Humana Press, © 2002, incorporated herein by reference.

Examples of fluorophores that can be used in the methods described herein are: 7-amino-4-methylcoumarin-3-acetic acid (AMCA), Texas Red™ (Molecular Probes, Inc., Eugene, Oreg.); 5-(and-6)-carboxy-X-rhodamine, lissamine rhodamine B, 5-(and-6)-carboxyfluorescein; fluorescein-5-isothiocyanate (FITC); 7-diethylaminocoumarin-3-carboxylic acid, tetramethylrhodamine-5-(and-6)-isothiocyanate; 5-(and-6)-carboxytetramethylrhodamine; 7-hydroxycoumarin-3-carboxylic acid; 6-[fluorescein 5-(and-6)-carboxamido]hexanoic acid; N-(4,4-difluoro-5,7-dimethyl-4-bora-3a,4a diaza-3-indacenepropionic acid; eosin-5-isothiocyanate; erythrosine-5-isothiocyanate; 5-(and-6)-carboxyrhodamine 6G; and Cascade™ blue acetylazide (Molecular Probes, Inc., Eugene, Oreg.).

Should multiple probes be used, e.g., for detecting CEN 7 and the EGFR gene, fluorophores of different colors can be chosen such that each chromosomal probe in the set can be distinctly visualized. Preferably a probe panel will comprise separate probes, each labeled with a different fluorophore.

Probes can be viewed with a fluorescence microscope and an appropriate filter for each fluorophore, or by using dual or triple band-pass filter sets to observe multiple fluorophores. See, e.g., U.S. Pat. No. 5,776,688 to Bittner, et al., which is incorporated herein by reference. Any suitable microscopic imaging method can be used to visualize the hybridized probes, including automated digital imaging systems, such as those available from MetaSystems or Applied Imaging. Alternatively, techniques such as flow cytometry can be used to examine the hybridization pattern of the chromosomal probes.

Probes can also be labeled indirectly, e.g., with biotin or digoxygenin by means well known in the art. However, secondary detection molecules or further processing are then required to visualize the labeled probes. For example, a probe labeled with biotin can be detected by avidin conjugated to a detectable marker, e.g., a fluorophore. Additionally, avidin can be conjugated to an enzymatic marker such as alkaline phosphatase or horseradish peroxidase. Such enzymatic markers can be detected in standard colorimetric reactions using a substrate for the enzyme. Substrates for alkaline phosphatase include 5-bromo-4-chloro-3-indolylphosphate and nitro blue tetrazolium. Diaminobenzidine can be used as a substrate for horseradish peroxidase.

The probes and probe sets useful with the methods of the invention can be packaged with other reagents into kits to be used in carrying out the methods of the invention. Useful probe sets and kits can comprise probes to CEN 7 and probes to one or more genetic loci such as EGFR and Her2.

Expression Reagents. Protein expression can be measured by IHC using antibody probes that specifically bind to proteins of interest, such as pAKT and PTEN. A wide range of antibody probes is available which includes the major cell signaling pathway components. Kits are also available that include the antibody probes and the detection reagents. The antibody probe may be labeled with fluorophores, enzymes, or moieties that allow additional binding of detection reagents (e.g., biotin that is bound further by a labeled avidin or streptavidin). Fluorescent antibodies are visualized directly under a fluorescence microscope while enzyme labels are incubated with substrate to produce insoluble chromogenic or fluorescent products that are visualized using a bright-field or fluorescence microscope, respectively. Indirectly labeled in situ hybridization probes, e.g., enzyme labels on antibodies, can be detected in standard colorimetric reactions using a substrate for the enzyme. Substrates for alkaline phosphatase include 5-bromo-4-chloro-3-indolylphosphate and nitro blue tetrazolium. Diaminobenzidine can be used as a substrate for horseradish peroxidase. The first antibody bound to the expressed protein may also be bound by a second antibody that specifically binds the first antibody (e.g. anti-mouse IgG). Expressed mRNA precursor to the protein may also be detected by in situ hybridization or by reverse-transcriptase polymerase chain reaction (PCR).

The probes and probe sets useful in the invention can be packaged with expression reagents into kits to be used in carrying out the methods of the invention. Useful kits can include antibody probes that specifically bind to proteins of interest, such as pAKT and PTEN.

Preparation of Samples. A biological sample is a sample that contains cells or cellular material. For example, lung samples are typically cells or cellular material derived from pulmonary structures, including but not limited to lung parenchyma, bronchioles, bronchial, bronchi, and trachea. Non-limiting examples of biological samples useful for the detection of lung cancer include bronchial specimens, resected lung, lung biopsies, and sputum samples. Examples of bronchial specimens include bronchial secretions, washings, lavage, aspirations, and brushings. Lung biopsies can be obtained by methods including surgery, bronchoscopy, fine needle aspiration (FNA), and transthoracic needle biopsy. In one example, touch preparations can be made from lung biopsies.

Tissues can be fixed with a fixative such as formaldehyde and then embedded in paraffin. Sections are then cut using a microtome and are applied to a microscope slide. Cytology specimens can be prepared from cellular suspensions derived from FNA, bronchial washings, bronchial lavage, or sputum, or disseminated tissue cells. Cytology specimens can be prepared by fixation of cells in ethanol or methanol:acetic acid combined with cytocentrifugation, thin layer deposition methods (e.g. ThinPrep, Cytyc Corp.), smears, or pipetting onto microscope slides.

In addition, biological samples can include effusions, e.g., pleural effusions, pericardial effusions, or peritoneal effusions. In addition, biological samples can include cells or cellular material derived from tissues to which lung cancers commonly metastasize. These tissues include, for example, lymph nodes, blood, brain, bones, liver, and adrenal glands. Thus, the probes and probes sets described herein can be used to detect lung cancer and lung cancer metastasis.

Head and neck samples are typically cells or cellular material derived from resected tumors and biopsies, and are otherwise prepared as for lung specimens

Pre-Selection of Cells. Cell samples can be evaluated preliminarily by a variety of methods and using a variety of criteria. The probes and methods described herein are not limited to usage with a particular screening methodology. One example is the “scanning method” wherein the observer scans hundreds to thousands of cells for cytologic abnormalities, e.g., as viewed with a DAPI filter. The number of cells assessed will depend on the cellularity of the specimen, which varies from patient to patient. Cytologic abnormalities commonly but not invariably associated with dysplastic and neoplastic cells include nuclear enlargement, nuclear irregularity, and abnormal DAPI staining (frequently mottled and lighter in color). In the scanning step, the observer preferably focuses the evaluation of the cells for chromosomal abnormalities (as demonstrated by FISH) to those cells that also exhibit cytological abnormalities. In addition, a proportion of the cells that do not have obvious cytologic abnormalities can be evaluated since chromosomal abnormalities also occur in the absence of cytologic abnormalities. This scanning method is described in further detail in U.S. Pat. No. 6,174,681 to Hailing, et al., which is incorporated herein by reference. Lung cancer cells can be selected for evaluation using the method described US Patent Pub. 2003/0087248 A1 by Morrison, et al., which is incorporated herein by reference.

Regions of the specimen may also be selected for evaluation using conventional stains, such as stains containing hematoxylin and eosin. For example, a pathologist can stain a section of a paraffin-embedded specimen with a hematoxylin/eosin stain, identify a region as probably cancerous by tissue morphology and staining pattern, and outline that region with a felt tip ink pen or glass scribe. The marked region is then transferred to the corresponding location on a serial section of the paraffin-embedded specimen with a glass scribe, and FISH is performed on that slide. Cells within the scribed region are then evaluated for FISH signals,

Detection of Chromosomal Abnormalities. Abnormal cells are characterized by aneusomy or, preferably, polysomy of chromosome 7. Aneusomy of chromosome 7 is assessed by examining the hybridization pattern of the chromosomal probe (e.g., the number of signals for each probe) in the cell, and recording the number of signals. Aneusomy is typically intended to mean abnormal copy number, either of the whole chromosome or a locus on a chromosome. Abnormal copy number includes both monosomy (one copy) and nullsomy (zero copies) of the autosomes, and greater than 2 copies. Test samples are typically considered “test positive” for polysomy of chromosome 7 when found to contain about 3.0 or more copies of chromosome 7 per cell. For example, the cut of for polysomy may be set at above 3.0 signals per cell and, in a preferred embodiment, the cut off for polysomy may be set at a range of about 3.5-4.0 signals per cell. However, sectioning of paraffin-embedded specimens (typically 4-6 μm) results in truncation of cell nuclei such that the number of FISH signals per cell will be somewhat lower than the actual number of copies in an intact nucleus. Therefore, thresholds for polysomy and loss of copies are set empirically to reflect optimal association with response or survival. A practical cutoff for polysomy may be set at about 3.6 CEN 7 signals per cell since this may provide a better correlation with response or survival, even though cells with 3 or 4 actual copies of CEN 7 may fall below the cutoff. In this case, the “normal” range may include low level polysomy and the “polysomy” range may include only higher levels of polysomy. Criteria for “test positive” can include testing positive with a CEN 7 probe depending upon the clinical correlation between the abnormal loci and patient response to therapy. When additional probes, such as probes to EGFR or Her2, are used test positive can include detection of abnormal hybridization patterns with a subset of probes. For example, the pattern of an initial subset of probes (e.g., the probe to CEN 7) can be assessed and, if appropriate, the test can be taken as positive without assessing the other probes.

Test samples can comprise any number of cells that is sufficient for a clinical diagnosis, and typically contain at least about 100 cells. In a typical assay, the hybridization pattern is assessed in about 20-200 cells. The number of cells identified with chromosomal abnormalities and used to classify a particular sample as positive in general will vary with the number of cells in the sample. The absolute number of cells detected with chromosomal abnormality or the percentage of the total number of cells examined that contain the abnormality, can be used to determine if a sample is positive by comparison to a cutoff value. If, for example, the number or percentage of cells with abnormality is equal to or below the cutoff value then the specimen can be classified as negative for the abnormality. If the number or percentage of cells with abnormality is greater than the cutoff value then the specimen can be classified as positive. Specimens positive for one or a particular set of chromosomal abnormalities can be classified as to the patient's probable response to medication. Alternatively, specimen positivity with respect to a chromosomal abnormality can be determined from the average copy number of a locus per cell in the specimen or the average ratio of one locus copy number to a second locus copy number for that specimen. Specimens having average copy numbers of a particular locus per cell above a cutoff established for abnormal gain of a locus, or below a cutoff established for abnormal loss of a locus are considered positive for the specific abnormality. Likewise cutoffs can be established for the relative gain or loss between two different loci and applied to the measured loci ratio to establish if a sample is positive or negative for that abnormality.

Protein Expression. Protein expression can be detected in tumor tissue, cell material obtained by biopsy and the like. For example, a biopsy sample can be immobilized and contacted with an antibody, an antibody fragment or an aptimer that binds selectively to the protein to be detected. The sample can be assayed to determine whether the antibody, fragment or aptimer has bound to the protein by techniques well known in the art. Protein expression can be measured by a variety of methods including but not limited to Western blot, immunoblot, enzyme-linked immunosorbant assay (ELISA), radioimmunoassay (RIA), immunoprecipitation, surface plasmon resonance, immunohistochemical (IHC) analysis, mass spectrometry, fluorescence activated cell sorting (FACS) and flow cytometry.

In a preferred embodiment, IHC analysis is used to measure protein expression. The level of expression for a sample is determined by IHC by staining the sample for a particular expression marker and developing a score for the staining. For example, rabbit monoclonal antibodies can be used to stain for the expression marker pAKT. Similarly mouse antibodies are known for use in the staining of the marker PTEN. Samples are evaluated for the frequency of cells stained for each sample and the intensity of the stain. Typically, a score based on the frequency (rated from 0-4) and intensity (rated from 0-4) of the stained sample is developed as a measure of overall expression. Exemplary but non-limiting methods for IHC and criteria for scoring expression are described in detail in Handbook of Immunohistochemistry and In Situ Hybridization in Human Carcinomas, M. Hayat Ed., 2004, Academic Press and are described in the examples. There, frequency and staining intensity were each rated from 0-4 and the product of intensity times frequency was taken to estimate overall expression. A score of 1-4 can be taken as an indication that the marker was positively expressed in the sample. Higher scores are used to indicate higher level expression.

Response to Therapy. Chromosomal probes and expression markers are chosen for the ability to classify patients as to response (or non response) to therapy when used in methods of the invention. Response to therapy is commonly classified by the RECIST criteria established by the World Health Organization, the National Cancer Institute and the European Organization for Research and Treatment of Cancer. The RECIST criteria classify response as progressive disease (PD), stable disease (SD), partial response (PR), and complete response (CR). Good response is typically considered to include PR+CR (collectively referred to herein as Objective Response).

Details of the invention are further described in the following examples, which are not intended to limit the scope of the invention as claimed. One of skill in the art will recognize that variations and modifications of the invention may be apparent upon reviewing the instant specification. It is therefore an object to provide for such modifications and variations of the embodiments described herein, without departing from the scope or the spirit of the invention.

### EXAMPLES

Experimental Methods

Specimens. Specimens from 81 Expanded Access Trial NSCLC patients treated more than one week with gefitinib (Iressa) were obtained from the archives of the Pathology Department of Rush University Medical Center and the University of Chicago (Chicago, Ill.). Chart review and study analyses were approved by the RUMC Institutional Review Board. The diagnosis of NSCLC in the archival material was obtained from pathology reports and confirmed by histologic evaluation before further analysis. Age, gender, smoking status and disease grade were established from chart review and patient report at registration. Smoking status was defined by lifetime consumption of <100 cigarettes. Response was assessed according to RECIST criteria of measurable and non-measurable lesions. Progression-free interval and overall survival were counted in months (days divided by 30.4) from the time of initial treatment with gefitinib. Progressive disease was defined by relapse within 70 days of treatment.

In Situ Hybridization. For copy number analyses by FISH, EGFR and centromere 7 (CEN7) probes were utilized to examine EGFR/cell, CEN7/cell and EGFR/CEN 7. A probe targeting α satellite repeat sequences near the centromere of chromosome 7 was used to indicate CEN 7 copy number (SpectrumGreen™ CEP® 7, Abbott Molecular Inc.). Specimen slides were prepared using either the Vysis Paraffin Pretreatment II or III kits (Abbott Molecular Inc.). The prepared specimen slides were hybridized with two-color FISH probe solutions (SpectrumOrange™ LSI® EGFR, Spectrum-Green CEP 7; Abbott Molecular Inc.) in a HYBrite™ automated co-denaturation oven (Abbott Molecular Inc.). The slides were placed on the oven surface and 10 μL probe solution was layered over the tissue section. A cover slip was applied over the probe solution and sealed to the slide with rubber cement. After denaturation at 73° C. for 5 minutes, the probe was hybridized at 37° C. for 16-18 hr. Following hybridization and removal of the rubber cement seal, the slides were placed in room-temperature 2×SSC (SSC=0.3 M NaCl, 15 mM sodium citrate), 0.3% Nonidet P40 (NP40) for 2-5 min to detach the cover slips. The slides were then immersed in 73° C. 2×SSC, 0.3% NP40 for 2 min to remove nonspecifically bound probe and then were allowed to dry in the dark. DAPI I antifade solution (Abbott Molecular Inc.) was applied to the specimen for visualization of the nuclei. Some of the specimens required additional processing to yield optimal FISH results. Over-digested or under-digested specimens were reprocessed as described previously.

The FISH slides were evaluated under a Zeiss Axioscope epi-fluorescence microscope (Carl Zeiss, Thornwood, N.Y.). Signals were visualized and counting performed with a DAPI single-band-pass filter set to visualize nuclei, an orange single-band-pass filter set to visualize the SpectrumOrange-labeled LSI EGFR probe and a green single-band-pass filter set to visualize the SpectrumGreen CEP 7 probe (all filter sets from Abbott Molecular Inc.). Only nuclei with morphology characteristic of malignant cells were counted.

Typically, 30-90 (median 80) cells were enumerated in each specimen. The mean number of signals per cell was calculated by totaling the number of signals from each cell for EGFR and CEN 7, and dividing by the number of cells counted to provide EGFR/cell and CEN 7/cell, respectively. EGFR/cell was divided by CEN 7/cell signals per cell to yield EGFR/CEN7. Other FISH parameters used in developing selection criteria are defined as follows. The EGFR % gain or CEN 7% gain was calculated as the percentage of cells with more than two EGFR or CEN 7 signals, respectively. EGFR/CEN 7% gain was the percentage of cells that showed more EGFR signals than centromere 7 signals. When a slide was counted multiple times, counts were combined and used for recalculating the ratios and % gain.

Optimal cutoff points for defining high ratios or high % gains were selected by first generating cutoffs from the mean minus 1.5 standard deviations to the mean plus 3.5 standard deviations, in 0.1 standard deviation increments, for each parameter (ratios and % gains), using the mean and standard deviations of the non responding patients. Each high and low ratios and % gains at each cutoff were compared with objective response and survival (greater than or less than 1 year survival) in contingency tables. Cutoffs with the lowest chi-square probabilities were selected for further analysis. A cutoff for CEN 7/cell near 3.6 was found to be optimal for defining chromosome 7 polysomy with respect to Objective Response.

Cutpoints were also assigned to indicate loss of chromosome 7, either monosomy or nullisomy. Chromosome 7 aneusomy (CEN 7 aneusomy) was then defined, for example, as CEN 7/cell below about 2.0 or above about 3.0 (preferably above about 3.6).

Immunohistochemistry. Paraffin sections (5 μm, freshly cut) were deparaffinized and rehydrated by standard technique. A microwave antigen retrieval method was then carried out in citrate buffer. The tissue was stained using a Ventana ES Histo-stainer (Ventana Medical Systems, Tucson, Ariz.) using supplied diaminobenzidine and avidin-biotin conjugate immunoperoxidase chemistry. Sections were stained for expression of markers listed in Table 1.

TABLE 1
Antibodies used for IHC staining

| Marker | Antibody | Staining pattern | Dilution |
|---|---|---|---|
| EGFR | M3563 mouse monoclonal antibody | Cell membrane | 1:200 |
| | (Dako Corp., Carpinteria, CA) | | |
| pAKT | 3787S rabbit monoclonal antibody | Cytoplasmic, | 1:40 |
| | (Cell Signalling Technology, | nuclear | |
| | Beverly, MA) | | |
| PTEN | MS-1797-S0 mouse monoclonal | Nuclear | 1:20 |
| (Clone | antibody (Lab Vision, Neomarkers,) | | |
| 28H6) | | | |

Immunostaining frequency of all tumor cells on each slide was estimated on a scale of 0 to 4 without knowledge of clinical patient data. Fewer than 1% positive tumor cells were scored as 0, 1% to 10% as 1, and 11-35% as 2, 36% to 70% as 3, and over 70% as 4. Tumor cell staining intensity was also scored on a scale of 0 to 4. The product of the intensity times the frequency, or the frequency alone, was used as a relative estimate of overall expression. Only cell-membrane-associated staining was considered for EGFR.

Statistical Methods. Univariate analysis of association between two variables was performed using the Fisher's Exact Test. Multivariate analysis of two or more markers was assessed by Chi Square. The level of significance was p<0.05 in one- or two-tailed estimates.

The Kaplan-Meier method was used to determine progression-free interval and overall survival, with comparison between groups assessed by log-rank test.

Results

Patients and Clinical Assessments. Eighty-one patients were selected for this study, based on their being treated in the gefitinib expanded access trial and tissue availability. The demographics of this patient group are shown in Table 2. All patients received 250 mg daily gefitinib with a median follow-up period of 7.3 months.

TABLE 2
Patient demographic characteristics and response to treatment

| | No. of | | |
|---|---|---|---|
| | patients | Objective | |
| Characteristic | (%) | Response* (%) | p value |
| Total | 81 (100) | 12 (15) | — |
| Age | | | 0.4621 |
| ≧60 years | 62 (77) | 4 (21) | |
| <60 years | 19 (23) | 8 (13) | |
| Gender | | | 1.000 |
| Male | 37 (46) | 5 (14) | |
| Female | 44 (54) | 7 (16) | |
| Smoking status | | | <0.001 |
| Yes | 69 (85) | 5 (7) | |
| Never smoked | 12 (15) | 7 (58) | |
| Histopathological | | | 0.3253 |
| subtype | | | |
| Bronchoalveolar, | 56 (69) | 10 (18) | |
| adenocarcinoma | | | |
| Other | 25 (31) | 2 (8) | |
| Performance status | | | 0.7528 |
| 0 to 1 | 46 (58) | 6 (13) | |
| 2 to 4 | 34 (42) | 6 (18) | |
| Prior chemotherapy | | | 0.8412 |
| None | 14 (17) | 2 (14) | |
| One | 39 (48) | 7 (18) | |
| Two or more | 28 (35) | 3 (5) | |

*Partial and complete response as defined by RECIST criteria

Overall response to gefitinib was 15% in this group of patients, including 2 patients with complete response and 10 with partial response. Thirty-six patients demonstrated stable disease.

Molecular Predictors of Response. Genotypic and phenotypic markers were analyzed in this patient group with accessible tumor tissue. Results for proposed predictors of response are shown in Table 3. The following markers were significantly associated with response to gefitinib: EGFR/cell ≧6.0 (p=0.0087), EGFR % gain ≧75% cells (p=0.0352), CEN 7/cell ≧4.0 (p=0.0294), and PTEN expression (PTEN IHC; p=0.0147).

TABLE 3
Molecular analysis of response in non-small cell lung cancer patients.*

| | | | Fishers |
|---|---|---|---|
| | Number of | Objective | Exact |
| Variable | Patients (%) | Response† (%) | p Value |
| EGFR IHC | | | 0.2158 |
| 0 | 35 (43) | 3 (9) | |
| 1+ to 4+ | 46 (57) | 9 (20) | |
| EGFR/cell \|\| | | | 0.0087 |
| ≦6 | 70 (86) | 7 (10) | |
| ≧6 | 11 (14) | 5 (46) | |
| EGFR % gain¶ | | | 0.0352 |
| <75% | 57 (70) | 5 (9) | |
| ≧75% | 24 (30) | 7 (29) | |
| EGFR/CEN 7 % gain¶ | | | 0.0667 |
| <34% | 41 (51) | 3 (7) | |
| ≧34% | 40 (49) | 9 (23) | |
| EGFR/CEN 7 | | | 0.1648 |
| <1 | 22 (27) | 1 (5) | |
| ≧1 | 59 (73) | 11 (19) | |
| CEN 7/cell¶ | | | |
| <3.6 | 63 (78) | 7 (11) | 0.1262 |
| ≧3.6 | 18 (22) | 5 (28) | |
| <3.8 | 65 (80) | 7 (11) | 0.0538 |
| ≧3.8 | 16 (20) | 5 (31) | |
| <4 | 67 (83) | 7 (11) | 0.029 |
| ≧4 | 14 (17) | 5 (36) | |
| <3.8 and >2.0 (“normal”) | 55 (68) | 7 (13) | 0.5089 |
| >3.8 or <2.0 (aneusomy) | 26 (32) | 5 (19) | |
| pAKT IHC# | | | 0.3268 |
| Absent | 39 (53) | 4 (10) | |
| Present | 34 (47) | 7 (21) | |
| PTEN IHC# | | | 0.0147 |
| Absent | 46 (63) | 3 (7) | |
| Present | 27 (37) | 8 (30) | |

*Data from evaluable tumors
†Partial or complete response according to RECIST criteria
¶Results from 81 patients evaluated by FISH as described in the Methods section
#Tissue sections from 73 patients were analyzed by IHC. PAKT expression and PTEN expression are defined as 1+ to 4+ staining by IHC.

The effect of interpretive criteria on the associations with response was observed. Chromosome 7 polysomy, as assessed by FISH using a centromeric probe, demonstrated a range of relationships to response, depending on the criteria used for interpretation of the raw data. When cutoffs of 3.0, 3.5, 3.6, 3.8 and 4.0 CEN 7/cell were used, p values were 0.420, 0.221, 0.079, 0.039 and 0.029 respectively. Although tumors carrying ≧4.0 signals per cell were more highly associated with response, a cutoff of about 3.6 was more predictive of survival.

Molecular Predictors of Survival. Results for proposed predictors of survival are shown in Table 4. In general, parameters involving EGFR copy number were not statistically significantly associated with longer survival, even when normalized to CEN 7. Likewise, protein expression, as measured by IHC, was not significantly associated with longer survival. However, parameters based on chromosome 7 copy number were highly associated, i.e., CEN 7/cell: “normal” versus aneusomy (p=0.001 8), polysomy versus non-polysomy (p=0.01 49), and the percentage of cells containing four or more copies of CEN 7 (p=0.0248).

TABLE 4
Molecular predictors of survival in NSCLC patients.

| | | Median | |
|---|---|---|---|
| | | Survival | |
| Variable | n | (Months) | Log-rank p |
| EGFR Expression, IHC | | | 0.6727 |
| Not detected | 35 | 8.5 | |
| Present | 46 | 7.1 | |
| CEN 7/cell | | | |
| <3.6 and >2.0 (“normal”) | 55 | 5.8 | 0.0018 |
| >3.6 or <2.0 (aneusomy) | 26 | 15.3 | |
| <3.6 (non-polysomy) | 63 | 6.0 | 0.0149 |
| ≧3.6 (polysomy) | 18 | 16.2 | |
| EGFR/CEN 7 % gain | | | 0.0779 |
| <34% | 41 | 6.0 | |
| ≧3.4% | 40 | 10.3 | |
| pAKT Expression, IHC | | | 0.0690 |
| Not detected | 39 | 5.8 | |
| Present | 34 | 10.5 | |
| PTEN Expression, IHC | | | 0.0699 |
| Not detected | 31 | 5.9 | |
| Present | 42 | 9.3 | |
| EGFR % gain¶ | | | 0.48 |
| <75% | 57 | 7.9 | |
| ≧75% | 24 | 9.4 | |
| CEN 7 % ≧4 copies | | | 0.0248 |
| <52% | 64 | 6.9 | |
| ≧52% | 17 | 17.1 | |

¶Results evaluated by FISH as described in the Methods section

EGFR and Chromosome Status Combined with pAKT and PTEN Expression as Predictors of Survival. Examples of marker combinations that were significantly associated with survival are shown in Table 5. In each of the examples listed, the combination of the two parameters provided greater statistical significance, as judged by lower p-values for median survival, than either parameter individually (compare to Table 4). It may be noted that while individual parameters based on EGFR copy number were not significant predictors of survival, the combination of some EGFR-based parameters with pAKT or PTEN expression did provide significant associations.

TABLE 5
EGFR and chromosome 7 status combined with
pAKT or PTEN expression as potential predictors of survival.

| | | Median | |
|---|---|---|---|
| | | Survival | Log-rank |
| Variable | n | (months) | p |
| EGFR % gain, pAKT | | | 0.0153 |
| ≧75% cells and *PAKT⁺ | 11 | 24.5 | |
| Any negative | 62 | 6.6 | |
| EGFR/CEN7 % gain, PTEN | | | 0.0090 |
| EGFR ≧33 *PTEN⁺ | 21 | 18.2 | |
| Any negative | 58 | 6.6 | |
| CEN 7/cell, pAKT | | | 0.0008 |
| CEN 7 ≧3.6 (polysomy), | 11 | 24.5 | |
| *PAKT⁺ | | | |
| Any negative | 62 | 5.9 | |
| CEN7 % ≧4 copies, pAKT | | | 0.0013 |
| ≧52%,* pAKT+ | 10 | 39.4 | |
| Any negative | 63 | 5.9 | |

*Expression measured by IHC stain frequency:
⁻= 0;
⁺= 1-4

Discussion of Results

Targeted cancer therapies such as the TKIs gefitinib and erlotinib and the anti-EGFR monoclonal antibody, cetuximab (Erbitux) are most effective against cells with a strong dependence on the therapeutic target (EGFR) for malignant growth. The genetic instability of neoplastic cells, however, can override specific inhibitors by generating resistance mutations, or alleviate EGFR dependence by developing alternate signaling pathways and growth requirements. A simple relationship between the therapeutic target and the tumor phenotype, therefore, can change over the course of the disease and the initial effect of disabling EGFR, under some circumstances, will not translate into a long-term survival effect. This is illustrated in the lack of significant survival benefits of gefitinib, despite initial response as well as the number of biomarkers reported to be associated with sensitivity to gefitinib.

For the group of 81 patients studied here, parameters involving EGFR were found to provide the best classification of patients relative to immediate response to the TKI drug gefitinib. However, single parameters based on EGFR did not provide statistically significant patient classification with respect to survival. By contrast, single parameters based on chromosome 7 copy number was less effective in classifying patients with respect to response, but provided the most statistically significant classification of patients with respect to survival.

In the group of patients studied, chromosome 7 polysomy, (optimally ≧about 3.6 CEN 7 signals per cell), identified a subgroup of 18 patients with 16.2 month median survival, compared to the remaining 63 patients with 6.0 month median survival (p=0.0149). Chromosome 7 aneusomy, (optimally ≧about 3.6 CEN 7/cell or <2.0 CEN 7/cell, identified a subgroup of 26 patients with 15.3 month median survival, compared to the remaining 55 patients with 5.8 month median survival (p=0.0018). Another measure of abnormal chromosome 7 copy number, the percentage of cells with ≧4 copies per cell, identified a subgroup of 17 patients with 17.1 month median survival, compared to the remaining 64 patients with 6.9 month median survival (p=0.0248).

In addition to genomic copy number changes, increased expression of various proteins, often measured by IHC, have been investigated as predictors of response to TKIs. In the present study, increased EGFR protein measured by IHC was not significantly related to response nor clinical outcome. Similar results have been reported from analysis of EGFR transcription using qPCR. In the present study, pAKT expression was also not found to be an effective predictor of response or survival, and PTEN showed significant association with response but not survival.

Consideration of multiple factors may enhance the ability to predict TKI efficacy. Addition of pAKT expression status to EGFR status or polysomy 7 status improved prediction of longer survival time for the CEN 7/cell, EGFR % gain, and CEN7% ≧4 parameters. Addition of PTEN expression status to the EGFR/CEN 7% gain parameter also improved prediction of longer survival time.

It is to be understood that, while the invention has been described in conjunction with the detailed description, thereof, the foregoing description is intended to illustrate and not limit the scope of the invention. Other aspects, advantages, and modifications of the invention are within the scope of the claims set forth below.

What is claimed is:

1. A method for identifying a candidate patient for treatment with an inhibitor of the tyrosine kinase activity of EGFR, the method comprising: (a) obtaining a biological sample from the patient; (b) contacting the sample with a probe able to detect the presence of chromosome 7 under conditions sufficient to enable hybridization of the probe to chromosome 7 in the sample, if any, wherein the probe is able to detect the copy number of chromosome 7; and (c) identifying the candidate as being suitable for treatment with such tyrosine kinase inhibitors by identifying samples with an abnormal copy number of chromosome 7 and correlating said sample with said candidate patient.

2. The method of claim 1, wherein the biological sample is further contacted with probes able to detect the presence of EGFR or Her2.

3. The method of claim 1 further comprising the step of determining the presence or absence of aneusomy of chromosome 7 in the sample.

4. The method of claim 1 further comprising the step of determining the presence or absence of polysomy of chromosome 7 in the sample.

5. The method of claim 4 further comprising the step of determining whether the average copy number of chromosome 7 in the patient sample is greater than about 3.0 copies per cell.

6. The method of claim 4 further comprising the step of determining whether the average copy number of chromosome 7 in the patient sample is in the range of about 3.5 to about 4.0 copies per cell.

7. The method of claim 1 further comprising the step of contacting a biological sample from the patient with expression reagents for determining the presence of pAKT expression.

8. The method of claim 7 further comprising the step of determining the expression level of pAKT.

9. The method of claim 1, wherein the biological sample comprises a biopsy.

10. The method of claim 1, wherein the biological sample comprises a cytology sample.

11. The method of claim 1, wherein the chromosomal probes are fluorescently labeled.

12. The method of claim 1, wherein the biological sample comprises lung cells.

13. The method of claim 1, wherein the candidate patient has been diagnosed with a lung cancer.

14. The method of claim 1 further comprising the step of treating the candidate with an inhibitor of the tyrosine kinase activity of EGFR.

15. The method of claim 14 wherein the inhibitor of the tyrosine kinase activity of EGFR is selected from the group gefitinib, erlotinib and cetuximab.

16. The method of claim 1, wherein the candidate patient has been diagnosed with NSCLC.

17. A method for identifying a candidate patient for treatment with an inhibitor of the tyrosine kinase activity of EGFR or an agent that functions similarly to tyrosine kinase inhibitors, the method comprising: (a) obtaining a biological sample from the patient; (b) contacting a set of one or more chromosomal probes under conditions sufficient to enable hybridization of the probes to chromosomes in the sample if any, wherein one probe is able to detect copy numbers Chromosome 7 in the cells; and (c) identifying the candidate as being suitable for treatment with an inhibitor of the tyrosine kinase activity of EGFR or an agent that functions similarly to tyrosine kinase inhibitors by identifying samples with an abnormal copy number of chromosome 7 and correlating said sample with said candidate patient.

18. The method of claim 17 wherein the tyrosine kinase inhibitor is selected from the group gefitinib, erlotinib and cetuximab.
